SEQUENCE LISTING (2 sequences)

SEQ ID NO: 1 - DNA, 960 bp, Homo sapiens
  CDS (1)..(960)
atgagtcagcaaaacaccagtggggactgcctgtttgacggtgtcaacgagctgatgaaaaccctacagtttgcagtccacatccccaccttcgtcctgggcctgctcctcaacctgctggccatccatggcttcagcaccttccttaagaacaggtggcccgattatgctgccacctccatctacatgatcaacctggcagtctttgacctgctgctggtgctctccctcccattcaagatggtcctgtcccaggtacagtcccccttcccgtccctgtgcaccctggtggagtgcctttacttcgtcagcatgtacggaagcgtcttcaccatctgcttcatcagcatggaccggttcttggccatccgttacccgctactggtgagccactccggtcccccaggaagatctttgggatctgcatgcacaatctgggtcctggtgtggaccggaagcatccctatctacagtttccatgggaaagtggaaaaatacatgtgcttccacaacatgtctgatgatacctggagcgccaaggtcttcttcccgctggaggtgtttggcttcctccttcccatgggcatcatgggcttctgctgctccaggagcatccacatcctgctgggccgccgagaccacacccaggactgggtgcagcagaaagcctgcatctacagcatcgcagccagcctggctgtattcgtggtctccttcctcccagtccacctggggttcttcctgcagttcctggtgagaaacagctttatcgtagagtgcagagccaagcagagcatcagcttcttcttgcaattgtccatgtgtttctccaatgtcaactgctgcctggatgttttctgctactactttgtcatcaaagaattccgcatgaacatcagggcccaccggccttccagggtccagctggtcctgcaggacaccacgatctcccggggctaa

SEQ ID NO: 2 - protein, 319 aa, Homo sapiens
  TRANSMEM (25)..(46)
  TRANSMEM (66)..(86)
  TRANSMEM (96)..(117)
  TRANSMEM (140)..(161)
  TRANSMEM (184)..(205)
  TRANSMEM (228)..(250)
  TRANSMEM (272)..(292)
MSQQNTSGDCLFDGVNELMKTLQFAVHIPTFVLGLLLNLLAIHGFSTFLKNRWPDYAATSIYMINLAVFDLLLVLSLPFKMVLSQVQSPFPSLCTLVECLYFVSMYGSVFTICFISMDRFLAIRYPLLVSHSGPPGRSLGSACTIWVLVWTGSIPIYSFHGKVEKYMCFHNMSDDTWSAKVFFPLEVFGFLLPMGIMGFCCSRSIHILLGRRDHTQDWVQQKACIYSIAASLAVFVVSFLPVHLGFFLQFLVRNSFIVECRAKQSISFFLQLSMCFSNVNCCLDVFCYYFVIKEFRMNIRAHRPSRVQLVLQDTTISRG

(19) United States
(12) Patent Application Publication
(10) Pub. No.: US 2003/0113814 A1
(43) Pub. Date: Jun. 19, 2003

(54) Identification of modulators of gpr55 activity

(76) Inventors: Andrew Brown, Stevenage (GB); Alan Wise, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/275,200

(22) PCT Filed: May 4, 2001
(86) PCT No.: PCT/GB01/01969

(51) Int. Cl. G01N 33/53; G01N 33/567

(52) U.S. Cl. 435/7.2

(57) ABSTRACT

A method for identification of an agent that modulates activity of G-protein coupled receptor 55 (GPR 55), which method comprises: (i) contacting a test agent with GPR 55 or a variant thereof which is capable of coupling to a G-protein; and (ii) monitoring for GPR 55 activity in the presence of a G-protein; thereby determining whether the test agent modulates GPR 55 activity.

### FIELD OF THE INVENTION

The present invention relates to the identification of modulators of G-protein coupled receptors, and the use of such modulators in the treatment of adipocyte associated conditions.

### BACKGROUND OF THE INVENTION

G-protein coupled receptors (GPCRs) are a super-family of membrane receptors that mediate a wide variety of biological functions. Upon binding of extracellular ligands, GPCRs interact with a specific subset of heterotrimeric G proteins that can, in their activated forms, inhibit or activate various effector enzymes and/or ion channels. All GPCRs are predicted to share a common molecular architecture consisting of seven transmembrane helices linked by alternating intracellular and extracellular loops. The extracellular receptor surface has been shown to be involved in ligand binding whereas the intracellular portions are involved in G protein recognition and activation.

Activation of receptors coupled to the Gᵢ family of G proteins leads to inhibition of adenylate cyclase and lowering of intracellular cAMP levels. In adipocytes this leads to inhibition of hormone-sensitive lipase (HSL) which regulates the process of lipolysis, i.e. the hydrolysis of triglycerides (TG) to glycerol and non-esterified fatty acids (NEFA). Inhibition of lipolysis and the concomitant lowering of NEFA levels cause a reduction of hepatic triglyceride synthesis resulting in a fall in the levels of TG-rich lipoproteins. This then leads to an elevation in high-density lipoprotein (HDL) levels, thus giving the desired clinical profile of high HDL and low TG for the treatment of dyslipidemia.

Furthermore, there are many epidemiological studies illustrating an inverse correlation between plasma HDL cholesterol and coronary artery disease. Many patients with decreased plasma HDL cholesterol levels also have elevated TG levels. Therefore an agent that inhibits adipocyte lipolysis, thereby reducing TG availability, may also result in an increase in plasma HDL cholesterol levels due to the equilibrium that exists between the levels HDL, LDL and triglycerides.

Adipocytes are known to express a number of Gᵢ-coupled receptors such as the adenosine A₁, prostaglandin EP3 and nicotinic acid receptors. Agonists at such GPCRs have been shown to be anti-lipolytic, i.e. they promote lipid lowering, and in the case of nicotinic acid have been used in the clinic to treat particular forms of dyslipidaemia. However, unlike the adenosine A₁ and EP3 receptors, the nicotinic acid receptor has yet to be identified at the molecular level.

### SUMMARY OF THE INVENTION

The present invention is based on the finding that expression of the G-protein coupled receptor, GPR 55, occurs principally in adipose tissue. GPR 55 was found to be highly expressed in breast adipose. It has also been found to be present in omental fat but not in subcutaneous adipose tissue. GPR 55 may therefore be used as a screening target for the identification and development of novel pharmaceutical agents for use inhibiting lipolysis. Accordingly the present invention provides a method for identification of an agent that modulates GPR 55 activity, which method comprises:

(i) contacting a test agent with a cell, such as an adipocyte, which expresses GPR 55 or a variant thereof which is capable of coupling to a G-protein; and

(ii) monitoring for GPR 55 activity in the presence of a G-protein; thereby determining whether the test agent modulates GPR 55 activity.

The test agent may be contacted in step (i) with cells that express GPR 55 or a variant thereof. Alternatively, the test agent may be contacted in step (i) with membrane obtained from such cells. The invention also provides:

a test kit suitable for identification of an agent that modulates GPR 55 activity, which kit comprises:

(a) GPR 55 or a variant thereof which is capable of coupling to a G-protein; and

(b) means for monitoring GPR 55 activity;

a method for identification of an agent that inhibits lipolysis, which method comprises contacting adipocytes in vitro with a test agent which modulates GPR 55 activity and which has been identified by the method of the invention and monitoring lipolysis, thereby determining whether the test substance is an inhibitor of lipolysis;

an activator of GPR 55 activity or an inhibitor of lipolysis identified by a method of the invention or a polynucleotide which encodes GPR 55 or a variant polypeptide, for use in a method of treatment of the human or animal body by therapy; and

use of such an activator, inhibitor or polynucleotide in the manufacture of a medicament for the treatment of dyslipidaemia and conditions associated with dyslipidaemia, coronary heart disease, atherosclerosis, thrombosis or obesity, angina, chronic renal failure, peripheral vascular disease, stroke, type II diabetes or metabolic syndrome (syndrome X).

The polynucleotide may comprise:

(a) the nucleotide sequence of SEQ ID NO: 1,

(b) a sequence which hybridizes under stringent conditions to the complement of SEQ ID NO: 1,

(c) a sequence that is degenerate as a result of the genetic code with respect to a sequence defined in (a) or (b), or

(d) a sequence having at least 60% identity to a sequence as defined (a), (b) or (c).

### BRIEF DESCRIPTION OF THE FIGURES

FIG. 1 illustrates the expression of GPR 55 in normal human tissues.

FIG. 2 shows the constitutive activity of a small number of orphan receptors including GPR 55 following transformation into yeast expressing the Gpa1/G_{α13} chimera.

FIG. 3 shows that AM251 acts as an agonist at GPR 55 following expression of GPR 55 in yeast containing Gpa1/G_{αI3}, G_{αi2} and G_{αi3} chimeras.

### BRIEF DESCRIPTION OF THE SEQUENCES

SEQ ID NO: 1 shows the DNA and amino acid sequences of human GPR 55.

SEQ ID NO: 2 is the amino acid sequence alone of GPR 55. The seven transmembrane domains are identified.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human G-protein coupled receptor, GPR 55, and variants thereof. GPR 55 has been cloned previously (Sawzdargo et al, Molecular Brain Research 64, 193-198, 1999). Sequence information for GPR 55 is provided in SEQ ID NO: 1 (nucleotide and amino acid) and in SEQ ID NO: 2 (amino acid). The invention can therefore use polypeptides consisting essentially of the amino acid sequence of SEQ ID NO: 2 or a functional variant of that sequence. A functional chimeric receptor containing a fragment of SEQ ID NO: 2 may therefore be used.

The term “variant” refers to a polypeptide which has the same essential character or basic biological functionality as GPR 55. The essential character of GPR 55 can be defined as that of a G-protein coupled receptor. GPR 55 couples to Gᵢ-protein. Thus, the term “variant” refers in particular to a polypeptide which activates Gᵢ.

To determine whether a candidate variant has the same function as GPR 55, the ability of the variant to activate Gᵢ-protein can be determined. The effect of the candidate variant on Gᵢ activation can be monitored. This can be carried out, for example, by contacting cells expressing the candidate variant with a ligand which activates Gᵢ-protein when contacted with cells that express GPR 55, and measuring a Gᵢ-coupled readout. A control experiment is typically also carried out in which cells of the same type as those expressing the candidate variant, but expressing GPR 55 instead, are contacted with the ligand and a corresponding Gᵢ-coupled readout is measured. The effect attained by the candidate variant can then be directly compared with that attained by GPR 55.

An alternative way to determine whether a variant polypeptide has the same function as GPR 55 is to determine whether the variant polypeptide binds to a ligand which activates Gᵢ when the ligand is contacted with GPR 55. Thus, the ligand should activate Gᵢ when contacted with cells that express GPR 55. The ability of a candidate variant to bind such a ligand can be determined directly by contacting the candidate variant with a radiolabelled ligand that binds to GPR 55 and monitoring binding of the ligand to the variant. Typically, the radiolabelled ligand can be incubated with cell membranes containing the candidate variant. The membranes can then be separated from non-bound ligand and dissolved in scintillation fluid to allow the radioactivity of the membranes to be determined by scintillation counting. Non-specific binding of the candidate variant may also be determined by repeating the experiment in the presence of a saturating concentration of non-radioactive ligand. Preferably a binding curve is constructed by repeating the experiment with various concentrations of the candidate variant. The ability to bind a ligand of GPR 55 may also be determined indirectly as described below.

Typically, polypeptides with more than about 65% identity, preferably at least 80% or at least 90% and particularly preferably at least 95%, at least 97% or at least 99% identity, with the amino acid sequence of SEQ ID NO: 1 or 2 over a region of at least 20, preferably at least 30, at least 40, at least 60 or at least 100 contiguous amino acids or over the full length of the amino acid sequence of SEQ ID NO: 1 or 2, are considered as GPR 55 variants. The UWGCG Package provides the BESTFIT program which can be used to calculate identity (for example used on its default settings) (Devereau et al (1984) Nucleic Acid Research 12, p387-395). The PILEUP and BLAST algorithms can be used to calculate identity or line up sequences (typically on their default settings), for example as described in Algschul S. F., (1993) J. Mol. Evol. 36: 290-300; Altschul, S. F. et al (1990) J. Mol. Biol. 215:-403-10. Software for performing BLAST analyses is publicly available through the National Centre for Biotechnology Information (http://www.ncbi.nlm.nih.gov/).

Variant polypeptides therefore include naturally occurring allelic variants. An allelic variant will generally be of human or non-human mammal origin, such as bovine or porcine origin. Alternatively, a variant polypeptide can be a non-naturally occurring sequence. A non-naturally occurring variant may thus be a modified version of GPR 55, i.e. a modified version of the polypeptide having the amino acid sequence of SEQ ID NO: 1 or 2.

The amino acid sequence of GPR 55 may be modified by deletion and/or substitution and/or addition of single amino acids or groups of amino acids as long as the modified polypeptide retains the capability to function as a G-protein coupled receptor. Such amino acid changes may occur in one, two or more of the intracellular domains of GPR 55 and/or one, two or more of the extracellular domains of GPR 55 and/or one, two or more of the transmembrane domains of GPR 55.

Amino acid substitutions may thus be made, for example from 1, 2, 3, 4 or 5 to 10, 20 or 30 substitutions. Conservative substitutions may be made, for example according to the following Table. Amino acids in the same block in the second column and preferably in the same line in the third column may be substituted for each other;

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

A variant polypeptide may be a shorter polypeptide. For example, a polypeptide of at least 20 amino acids or up to 50, 60, 70, 80, 100 or 150 amino acids in length may constitute a variant polypeptide as long as it demonstrates the functionality of GPR 55. A variant polypeptide may therefore lack one, two or more intracellular domains and/or one, two or more extracellular domains and/or one, two or more transmembrane domains. A variant polypeptide may thus be a fragment of the full length polypeptide. A shortened polypeptide may comprise a ligand-binding region (N-terminal extracellular domain) and/or an effector binding region (C-terminal intracellular domain). Such fragments can be used to construct chimeric receptors preferably with another 7-transmembrane G-coupled receptor.

Variant polypeptides include polypeptides that are chemically modified, e.g. post-translationally modified. For example, such variant polypeptides may be glycosylated or comprise modified amino acid residues. They may also be modified by the addition of histidine residues, for example 6 or 8 His residues, or an epitope tag, for example a T7, HA, myc or flag tag, to assist their purification or detection. They may be modified by the addition of a signal sequence to promote insertion into the cell membrane.

The invention also utilises nucleotide sequences that encode GPR 55 or variants thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NO: 1. Such nucleotides can be isolated from human cells or synthesised according to methods well known in the art, as described by way of example in Sambrook et al, Molecular Cloning: A Laboratory Manual, 2nd edition, Cold Spring Harbour Laboratory Press, 1989. Typically a useful polynucleotide comprises a contiguous sequence of nucleotides which is capable of hybridising under selective conditions to the coding sequence or the complement of the coding sequence of SEQ ID NO: 1.

A polynucleotide can hydridize to the coding sequence or the complement of the coding sequence of SEQ ID NO: 1 at a level significantly above background. Background hybridisation may occur, for example, because of other cDNAs present in a cDNA library. The signal level generated by the interaction between a polynucleotide and the coding sequence or complement of the coding sequence of SEQ ID NO: 1 is typically at least 10 fold, preferably at least 100 fold, as intense as interactions between other polynucleotides and the coding sequence of SEQ ID NO: 1. The intensity of interaction may be measured, for example, by radiolabelling the probe, e.g. with ³²P. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.003M sodium citrate at about 60° C.).

The coding sequence of SEQ ID NO: 1 may be modified by one or more nucleotide substitutions, for example from 1, 2, 3, 4 or 5 to 10, 25, 50 or 100 substitutions. The polynucleotide of SEQ ID NO: 1 may alternatively or additionally be modified by one or more insertions and/or deletions and/or by an extension at either or both ends. The modified polynucleotide generally encodes a polypeptide which has G-protein coupled receptor activity or inhibits the activity of GPR 55. Degenerate substitutions may be made and/or substitutions may be made which would result in a conservative amino acid substitution when the modified sequence is translated, for example as shown in the Table above.

A nucleotide sequence which is capable of selectively hybridising to the complement of the DNA coding sequence of SEQ ID NO: 1 will generally have at least 60%, at least 70%, at least 80%, at least 90%, at least 95%, at least 98% or at least 99% sequence identity to the coding sequence of SEQ ID NO: 1 over a region of at least 20, preferably at least 30, for instance at least 40, at least 60, more preferably at least 100 contiguous nucleotides or most preferably over the full length of SEQ ID NO: 1. Methods of measuring nucleic acid and protein homology are well known in the art. For example the UWGCG Package provides the BESTFIT program which can be used to calculate homology (Devereux et al 1984). Similarly the PILEUP and BLAST algorithms can be used to line up sequences (for example are described in Altschul 1993, and Altschul et al 1990). Many different settings are possible for such programs. In accordance with the invention, the default settings may be used.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

Polynucleotides may be used as a primer, eg a PCR primer or a primer for an alternative amplification reaction of a probe, eg labelled with a revealing label by conventional means for identifying mutations in GPR 55 that may be implicated in diseases resulting from abnormal lipolysis. Fragments of polynucleotides may be fused to the coding sequence of other proteins, preferably other G-protein coupled receptors, to form a sequence coding for a fusion protein.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500 nucleotides in length, or even up to a few nucleotides, such as five or ten nucleotides, short of the coding sequence of SEQ ID NO: 1.

The polynucleotides have utility in production of GPR 55 or variant polypeptides, which may take place in vitro, in vivo or ex vivo. The polynucleotides may be used as therapeutic agents in their own right, in gene therapy techniques. The polynucleotides are cloned into expression vectors for these purposes. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to a person skilled in the art. By way of further example in this regard we refer to Sambrook et al.

Expression vectors comprise a polynucleotide encoding the desired polypeptide operably linked to a control sequence which is capable of providing for the expression of the coding sequence by a host cell. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of RNA or DNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters, in particular adipose cell specific promoters are especially preferred. Viral promoters may al so be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide which comprise sequences homologous to eukaryotic genomic sequences, preferably mammalian genomic sequences, or viral genomic sequences. This will allow the introduction of the relevant polynucleotides into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Retrovirus vectors for example may be used to stably integrate the polynucleotide into the host genome. Replication-defective adenovirus vectors by contrast remain episomal and therefore allow transient expression.

Cells are transformed or transfected with the vectors to express the GPR 55 polypeptide or a variant thereof. Such cells may be eucaryotic or prokaryotic. They include transient or, preferably, stable higher eukaryotic cell lines such as mammalian cells or insect cells, lower eukaryotic cells such as yeast, and prokaryotic cells such as bacterial cells. Particular examples of cells which may be used to express GPR 55 or a variant polypeptide include mammalian HEK293T, CHO, HeLa and COS7 cells. Preferably the cell line selected will be one which is not only stable, but also allows for mature glycosylation and cell surface expression of the GPR 55 polypeptide or a variant. Cells such as adipocytes expressing the GPR 55 receptor or a variant polypeptide may be used in screening assays. Expression may be achieved in transformed oocytes. The GPR 55 polypeptide or a variant may be expressed in cells such as adipose tissue of a transgenic non-human animal, preferably a rodent such as a mouse.

The present invention is concerned in particular with the use of GPR 55 or a functional variant in screening methods to identify agents that may act as modulators of GPR 55 receptor activity and, in particular, agents that may act as modulators of lipolysis. Such modulators are useful in the treatment of dyslipidaemia, coronary artery disease, atherosclerosis, obesity and thrombosis, angina, chronic renal failure, peripheral vascular disease, stroke, type II diabetes and metabolic syndrome (syndrome X).

Any suitable form of assay may be employed to identify a modulator of GPR 55 activity and/or of lipolysis. In general terms, such screening methods involve contacting GPR 55 or a variant polypeptide with a test compound and then determining receptor activity. G-protein activation, and especially Gᵢ-protein activation, may be determined therefore. Where a test compound affects receptor activity, its effect on lipolysis can be determined by contacting adipocytes in culture with the test compound and measuring lipolysis.

Modulator activity can be determined in vitro or in vivo by contacting cells expressing GPR 55 or a variant polypeptide with an agent under test and by monitoring the effect mediated by the GPR 55 or variant polypeptide. Thus, a test agent may be contacted with isolated cells which express GPR 55 or a variant polypeptide. The cells may be provided in culture. Cells may be disrupted and cell membranes isolated and used.

The GPR 55 or variant polypeptide may be naturally or recombinantly expressed. Preferably, an assay is carried out in vitro using cells expressing recombinant polypeptide or using membranes from such cells. Suitable eucaryotic and procaryotic cells are discussed above. Preferably adipocytes are used.

Typically, receptor activity is monitored by measuring a Gᵢ-coupled readout. Gᵢ-coupled readout can be monitored using an electrophysiological method to determine the activity of G-protein regulated Ca²⁺ or K⁺ channels or by using fluorescent dye to measure changes in intracellular Ca²⁺ levels. Other methods that can typically be used to monitor receptor activity involved measuring levels of or activity of GTPγS or cAMP.

A standard assay for measuring activation of the Gᵢ family of G proteins is the GTP_{γ}S binding assay. Agonist binding to G protein-coupled receptors promotes the exchange of GTP for GDP bound to the α subunit of coupled heterotrimeric G proteins. Binding of the poorly hydrolysable GTP analogue, [³⁵S]GTP_{γ}S, to membranes has been used extensively as a functional assay to measure agonism at a wide variety of receptors. Furthermore, the assay is largely restricted to measuring function of receptors coupled to the Gᵢ family of G proteins due to their ability to bind and hydrolyse guanine nucleotide at significantly higher rates than members of the G_{q}, Gₛ and G₁₂ families. See Wieland and Jakobs, Methods Enzymol. 237, 3-13, 1994.

Yeast assays may be used to screen for agents that modulate the activity of GPR 55 or variant polypeptides. A typical yeast assay involves heterologously expressing GPR 55 or a variant polypeptide in a modified yeast strain containing multiple reporter genes, typically FUS1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains may also contain further gene deletions, such as deletions of SST2 and FAR1, to potentiate the assay. Ligand activation of the heterologous receptor can be monitored for example either as cell growth in the absence of histidine or with a suitable substrate such as beta-galactosidase (lacZ).

Alternatively melanophore assays may be used to screen for activators of GPR 55. GPR 55 or a variant polypeptide can be heterologously expressed in Xenopus laevis melanophores and their activation can be measured by either melanosome dispersion or aggregation. Basically, melanosome dispersion is promoted by activation of adenylate cyclase or phospholipase C, i.e. Gₛ and G_{q} mediated signalling respectively, whereas aggregation results from activation of Gᵢ-protein resulting in inhibition of adenylate cyclase. Hence, ligand activation of the heterologous receptor can be measured simply by measuring the change in light transmittance through the cells or by imaging the cell response.

Preferably, control experiments are carried out on cells which do not express GPR 55 or a variant polypeptide to establish whether the observed responses are the result of activation of the GPR 55 or the variant polypeptide.

In vitro assay systems to measure lipolysis include cell lines that can be induced to differentiate into adipocytes such as 3T3-L1(murine) and SAOS-2(human) cells (Imamura et al, J. Biol. Chem. 274, 33691-33695, 1999; Diascro et al, J. Bone & Mineral Res. 13, 96-106, 1998). Alternatively, primary adipocytes harvested from an animal or human donor may be used.

Additional assays may thus be carried out in adipocytes. For example, the hydrolysis of triglycerides (TG) to non-esterified fatty acids (NEFA) and glycerol is performed by hormone-sensitive lipase (HSL). The activity of HSL is regulated by cAMP-dependent protein kinases. Therefore, inhibition of cAMP generation by adenylate cyclase via Gᵢ-coupled receptors (e.g. GPR 55 or a variant thereof) results in the reduction of NEFA and glycerol levels generated by adipocytes. Chromogenic assays for both NEFA and glycerol are commercially available (Randox) and can be used to verify that pre-treatment of adipocytes with an agonist for GPR 55 results in a reduction in the levels of NEFA and glycerol derived from adipocytes. In addition, assays can be performed to measure the cAMP content of adipocytes in the presence and absence of modulators for GPR 55 or a variant thereof in order to correlate reduction in the products of lipolysis with the activation of a Gi-coupled receptor.

A standard method for identifying lipolysis inhibitors is as follows. Adipocytes, for example approximately 100,000 in 0.5 ml, are pre-treated with an agent under test. The pre-treated adipocytes are incubated in the presence of adenosine deaminase, thereby to prevent accumulation of endogenous adenosine. Incubation can be carried out for 30 minutes at 37° C. Cells are centrifuged and buffer withdrawn from below the cell layer, heated such as at 70° C. for 10 minutes and glycerol can be assayed enzymatically. A suitable assay method is described in McGowan et al, Clin. Chem. 29, 538-543, 1983).

Suitable test substances which can be tested in the above assays include combinatorial libraries, defined chemical entities, peptide and peptide mimetics, oligolnucleotides and natural product libraries, such as display (e.g. phage display libraries) and antibody products. In a preferred embodiment, the test substance is a nicotinic acid (Niacin). Assays may also be carried out using known ligands of other G-protein coupled receptors to identify ligands which act as agonists at GPR 55.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1μM to 10 μM.

Agents which modulate GPR 55 activity and which have been identified by assays in accordance with the invention can be used in the treatment or prophylaxis of lipid disorders which are responsive to regulation of GPR 55 receptor activity. Agents which activate GPR 55 receptor activity and/or which have been identified as inhibitors of lipolysis are preferred. In particular, such agents may be used in the treatment of dyslipidaemia and conditions associated with dyslipidaemia such as atherosclerosis, obesity, thrombosis or coronary artery disease, angina, chronic renal failure, peripheral vascular disease, stroke, type II diabetes, and metabolic syndrome (syndrome X).

The agents may be formulated with a pharmaceutically acceptable carrier and/or excipient as is routine in the pharmaceutical art. See for example Remington's Pharmaceutical Sciences, Mack Publishing Company, Eastern Pennsylvania 17^{th} Ed. 1985. The carrier or excipient may be an isotonic saline solution but will depend more generally upon the particular agent concerned and the route by which the agent is to be administered.

The agents may be administered by enteral or parenteral routes such as via oral, buccal, anal, pulmonary, intravenous, intra-arterial, intramuscular, intraperitoneal, topical or other appropriate administration routes. A therapeutically effective amount of a modulator is administered to a patient. The dose of a modulator may be determined according to various parameters and especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Alternatively agents which up-regulate GPR 55 expression or nucleic acid encoding GPR 55 or a variant polypeptide may be administered to the mammal. Nucleic acid, such as RNA or DNA, preferably DNA, is provided in the form of a vector, which may be expressed in the cells of a human or other mammal under treatment. Preferably such up-regulation or expression following nucleic acid administration will enhance GPR 55 activity.

Nucleic acid encoding the GPR 55 or variant polypeptide may be administered to a human or other mammal by any available technique. For example, the nucleic acid may be introduced by injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to the mucosal surfaces for example by intranasal, oral, intravaginal, intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

Polynucleotides encoding GPR 55 or a variant polypeptide can also be used to identify mutation(s) in GPR 55 genes which may be implicated in human disorders. Identification of such mutation(s) may be used to assist in diagnosis of dyslipidaema and conditions associated with dyslipidaemia such as, atherosclerosis, obesity, thrombosis, angina, chronic renal failure, peripheral vascular disease, stroke, type II diabetes, and metabolic syndrome (syndrome X) or other disorders or susceptibility to such disorders and in assessing the physiology of such disorders.

Antibodies (either polyclonal or preferably monoclonal antibodies, chimeric, single chain, Fab fragments) which are specific for the GPR 55 polypeptide or a variant thereof can be generated. Such antibodies may for example be useful in purification, isolation or screening methods involving immunoprecipitation techniques and may be used as tools to elucidate further the function of GPR 55 or a variant thereof, or indeed as therapeutic agents in their own right. Such antibodies may be used to block ligand binding to the receptor. A variety of protocols for competitive binding or immunoradiometric assays to determine the specific binding capability of an antibody are well known in the art (see for example Maddox et al, J. Exp. Med. 158, 1211 et seq, 1993).

The following Examples illustrate the invention.

### EXAMPLE 1

Tissue Distribution Analysis

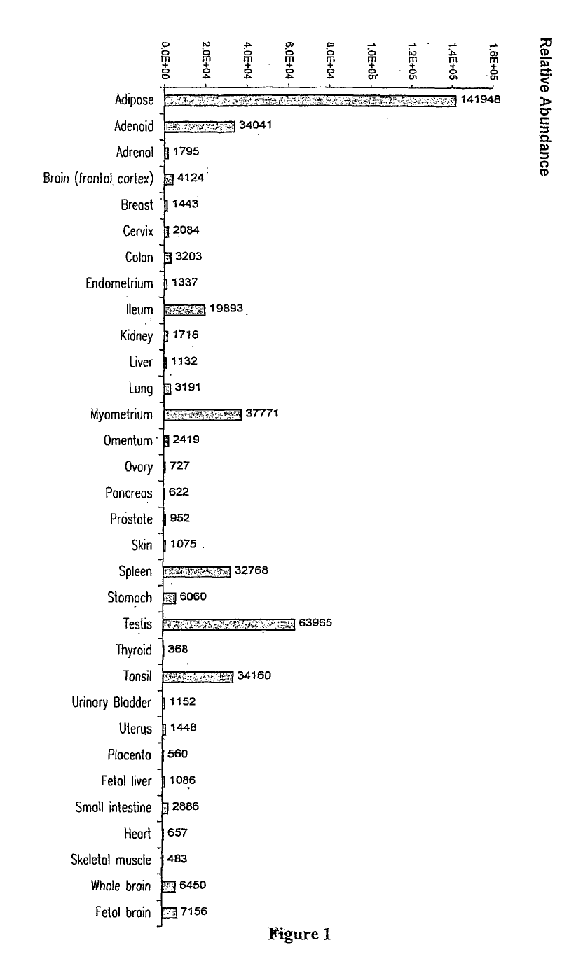

Taqman™ distribution analysis of GPR 55 was carried out to study expression of GPR 55 in normal human tissues. The results are shown in FIG. 1. These demonstrate that GPR 55 expression is essentially restricted to adipose tissue.

### Example 2

Tissue Distribution Analysis

The adipose tissue analysed in Example 1 was from the breast. Further Taqman™ distribution analysis of GPR 55 was carried out to study expression of GPR 55 in other types of normal human adipose tissue. High levels of expression of GPR 55 were found in omental adipose tissue but not in subcutaneous adipose tissue.

### Example 3

Expression and Screening Assay

Mammalian cells, such as HEK293, CHO and COS7 cells, over-expressing GPR 55 or a variant polypeptide are generated for use in the assay. 96 and 384 well plate, high throughput screens (HTS) are employed using fluorescence based calcium indicator molecules, including but not limited to dyes such as Fura-2, Fura-Red, Fluo 3 and Fluo 4 (Molecular Probes). Secondary screening involves the same technology. Tertiary screens involve the study of modulators in rat, mouse and guinea-pig models of disease relevant to the target.

A screening assay may be conducted as follows. Mammalian cells stably over-expressing the relevant polypeptide are cultured in black wall, clear bottom, tissue culture-coated 96 or 384 well plates with a volume of 100 μl cell culture medium in each well 3 days before use in a FLIPR (Fluorescence Imaging Plate Reader—Molecular Devices). Cells are incubated with 4 μM FLUO-3AM at 30° C. in 5% CO₂ for 90 mins and are then washed once in Tyrodes buffer containing 3 mM probenecid. Basal fluorescence is determined prior to addition of agents to be tested. The GPR 55 or variant polypeptide is activated upon the addition of a known agonist. Activation results in an increase in intracellular calcium which can be measured directly in the FLIPR. For antagonist studies, test agents are preincubated with the cells for 4 minutes following dye loading and washing and fluorescence is measured for 4 minutes. Agonists are then added and cell fluorescence measured for a further 1 minute.

Xenopus oocyte expression may be determined as follows. Adult female Xenopus laevis (Blades Biologicals) are anaesthetised using 0.2% tricaine (3-aminobenzoic acid ethyl ester), killed and the ovaries rapidly removed. Oocytes are then de-folliculated by collagenase digestion (Sigma type I, 1.5 mg ml⁻¹) in divalent cation-free. OR2 solution (82.5 mM NaCl, 2.5 mM KCl, 1.2 mM NaH₂PO₄, 5 mM HEPES; pH 7.5 at 25° C.). Single stage V and VI oocytes are transferred to ND96 solution (96 mM NaCl, 2 mM KCl, 1 mM MgCl₂, 5 mM HEPES, 2.5 mM sodium pyruvate; pH 7.5 at 25° C.) which contains 50 μg ml⁻¹ gentamycin and are stored at 18° C.

The GPR 55 receptor (in pcDNA₃, Invitrogen) is linearised and transcribed to RNA using T7 (Promega Wizard kit). m′G(5′)pp(5′)GTP capped cRNA is injected into oocytes (20-50 ng per oocyte) and whole-cell currents are recorded using two-microelectrode voltage-clamp (Geneclamp amplifier, Axon instruments Inc.) 3 to 7 days post-RNA injection. Microelectrodes have a resistance of 0.5 to 2MΩ when filled with 3M KCl.

### Example 4

Identification of Agonist Modulators of GPR55

Transformation of Yeast Assay Strains With Construct p426GPD-GPR55.

A system of yeast strains MMY14-MMY24 has been described previously (Olesnicky et al, EMBO J. 18, 2756-2763, 1999, Brown et al, Yeast 16, 11-22, 2000). These strains contain a series of genetic modifications to enable coupling of heterologously expressed receptors to the expression of two reporter genes, via the endogenous yeast pheromone response signal transduction pathway. Importantly, the gene encoding the endogenous yeast pheromone receptor, STE2, has been deleted from these strains such that cells containing p426GPD-GPR55 will express GPR55 protein in place of Ste2 receptor protein. Furthermore, the gene encoding the G-protein α-subunit involved in the pheromone response, GPA1, has been deleted from these strains. To enable receptor coupling in strains MMY14-24, plasmid constructs encoding modified versions of GPA1 have been stably integrated into the yeast chromosome and are expressed in place of endogenous yeast GPA1. The series of plasmids encoding modified versions of GPA1 has been described previously in WO 99/14344. Generally, the modifications made to Gpa1 facilitate coupling of heterologously expressed receptors to the yeast pheromone response pathway. The nature of the G proteins in these strains is given in the table below. The yeast strains were transformed with an expression plasmid p426GPD-GPR55 according to the routine methods (Gietz et al., Nucleic Acids Research 20, 1425, 1992).

TABLE
Yeast strains used in this experiment:

| | Gα chimera | |
|---|---|---|
| Yeast strain | species | Nature of Gα subunit |
| MMY14 | Gpa1/G_{αq} | “Transplant” Chimera (5 C-terminal amino |
| | | acids of Gpa1 replaced by mammalian Gα) |
| MMY15 | Gpa1/G_{αs} | Chimera (transplant) |
| MMY16 | Gpa1/G_{α16} | Chimera (transplant) |
| MMY17 | Gpa1/H_{α0} | Chimera (42 C-terminal amino acids of Gpa1 |
| | | replaced by mammalian Gα) |
| MMY19 | Gpa1/G_{α12} | Chimera (transplant) |
| MMY20 | Gpa1/G_{α13} | Chimera (transplant) |
| MMY21 | Gpa1/G_{α14} | Chimera (transplant) |
| MMY22 | Gpa1/G_{α0} | Chimera (transplant) |
| MMY23 | Gpa1/G_{α12} | Chimera (transplant) |
| MMY24 | Gpa1/G_{α13} | Chimera (transplant) |

Assay for Induction of Reporter Genes FUS1-lacZ and FUS1-HIS3 in response to GPR55 Ligands.

In vivo assays of reporter gene induction were carried out by suspending yeast cells transformed as described above to a density of 0.02 OD₆₀₀/ml in 200 μl SC-glucose (2%) medium lacking tryptophan, uracil and histidine. This medium was supplemented with 10 mM 3-aminotriazole and the β-galactosidase (lacZ) substrate chlorophenolred-β-D-galactopyranoside (CPRG; Boehringer Mannheim) to a concentration of 0.1 mg/ml. Additionally the medium was supplemented with various concentrations of the agonist ligand, AM251. To visualise the yellow to red colour change reaction occurring on degradation of CPRG due to β-galactosidase, the medium was buffered to pH 7 with 0.1 M sodium phosphate. The assay was conducted in flat-bottomed sterile 96-well microtitre plates. Plates were incubated for 24 hours at 30° C. without agitation, and absorbance at 570 nm was determined using a Spectrofluor microtitre plate reader (Tecan).

Demonstration of Functional Expression of Human GPR55 in the Yeast Saccharomyces cerevisiae and Coupling of GPR55 to the Yeast Pheromone Response Pathway.

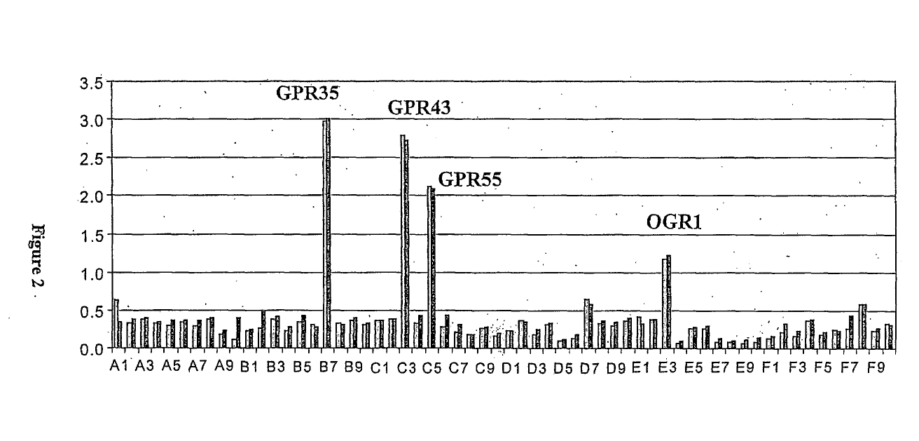

We found that GPR55 could be expressed in the yeast Saccharomyces cerevisiae and successfully coupled to the pheromone response pathway. Absorbance at 570 nm, corresponding to induction of FUS1-lacZ and FUS1-HIS3 reporter genes, was detected for cells containing p426GPD-GPR55 in combination with Gpa1/G_{α13} which was significantly higher than that seen in Gpa1/G_{α13} cells transformed with a vector only. These cells express a Gα subunit identical to Gpa1 but in which the 5 C-terminal amino acids are replaced with the 5 C-terminal amino acids of the mammalian Gα subunit, G_{α13}. This elevated basal response is observed in the absence of potential receptor modulators and, hence, can be termed “constitutive”. FIG. 2 shows basal responses in Gpa1/G_{α13}-containing yeast cells transformed to express a variety of orphan GPCRs. These data show that only a subset of receptors including GPR55 demonstrate constitutive activity following expression in this yeast strain.

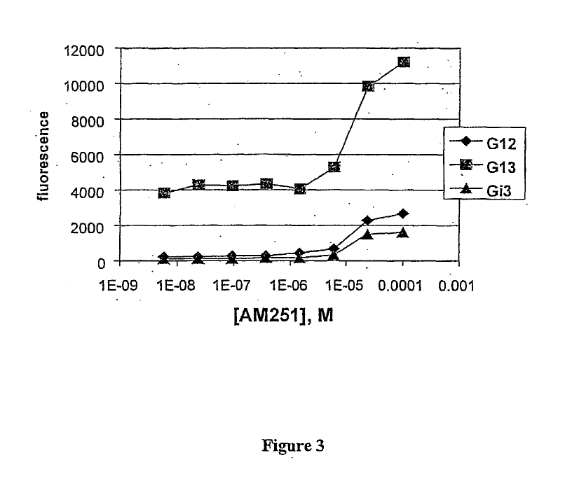

Addition of the cannabinoid CB1 receptor antagonist AM251 (N-(piperidin-1-yl)-5-(4-iodophenyl)-1-(2,4-dichlorophenyl)-4-methyl-1H-pyrazole-3-carboxamide) led to a significant increase in reporter gene activity in yeast co-expressing p426GPD-GPR55 and Gpa1/G_{α13}. The extent of this response was dependent on the concentration of AM251 (FIG. 3) and was not observed in control cells transformed with the vector p426GPD in combination with Gpa1/G_{α13} and therefore lacking GPR55 (data not shown). Analogues of this compound and other CB ligands available locally were tested at GPR55; the close analogue AM281 (1-(2,4-dichlorophenyl)-5-(4-iodophenyl)-4-methyl-N-4-morpholinyl-1H-pyrazole-3-carboxamide) also appeared active (data not shown). GPR55 was retransformed into a set of yeast strains expressing different Gpa1/G_{α13} chimeras and the activity of AM251 reproduced. AM251 very weakly activated unrelated receptors (EDG1; 1.3-fold over background) but the activation of GPR55 was much greater (up to 60-fold over background). The GPR55/AM251 activity has been consistently reproduced in cells expressing the Gpa1/G_{α13}. Activity has also been observed with Gpa1/G_{α12} and weaker activities in strains expressing Gpa1/G_{αi2} or Gpa1/G_{αi3} (FIG. 3).

1. A method for identification of an agent that modulates activity of G-protein coupled receptor 55 (GPR 55), which method comprises:
(i) contacting a test agent with GPR 55 or a variant thereof which is capable of coupling to a G-protein; and
(ii) monitoring for GPR 55 activity in the presence of a G-protein; thereby determining whether the test agent modulates GPR 55 activity.

2. A method according claim 1 wherein the test agent is contacted in step (i) with cells that express GPR 55 or a said variant thereof.

3. A method according to claim 1 wherein the test agent is contacted in step (i) with the membrane of cells that express GPR 55 or a said variant thereof.

4. A method according to claim 2 or 3 wherein the cells are adipocytes.

5. A method according to claim 4 wherein the adipocytes are provided as a differentiated cell line.

6. A method according to claim 4 wherein the adipocytes are primary adipocytes harvested from a human or animal donor.

7. A method according to any one of the preceding claims wherein the variant has at least 80% sequence identity to SEQ ID NO: 2.

8. A method according to any one of the preceding claims wherein the G-protein is G₁-protein.

9. A method according to claim 8 wherein step (ii) comprises determining whether Gᵢ-protein is activated.

10. A test kit suitable for identification of an agent that modulates GPR 55 activity, which kit comprises:
(a) GPR 55 or a variant thereof which is capable of coupling to a Gᵢ-protein; and
(b) means for monitoring GPR 55 activity.

11. A kit according to claim 10 wherein component (a) comprises cells which express GPR 55 or a said variant thereof.

12. A kit according to claims 10 or 11 wherein component (b) comprises means for determining whether Gᵢ-protein is activated.

13. A method for identification of an agent that inhibits lipolysis, which method comprises contacting adipocytes in vitro with a test agent identified by the method of any one of claims 1 to 9 and monitoring lipolysis, thereby determining whether the test agent is an inhibitor of lipolysis.

14. An activator of GPR 55 activity identified by a method according to any one of claims 1 or 9, an inhibitor of lipolysis identified by a method according to claim 13 or a polynucleotide which encodes GPR 55 or a variant polypeptide as defined in claim 1, for use in a method of treatment of the human or animal body by therapy.

15. An activator, inhibitor or polynucleotide according to claim 14 for use in the treatment of dyslipidaemia, coronary heart disease, atherosclerosis, thrombosis or obesity, angina, chronic renal failure, peripheral vascular disease, stroke, type II diabetes or metabolic syndrome (syndrome X).

16. A polynucleotide according to claim 14 or 15 comprising
(a) the nucleotide sequence of SEQ ID NO: 1,
(b) a sequence which hybridizes under stringent conditions to the complement of SEQ ID NO: 1,
(c) a sequence that is degenerate as a result of the genetic code with respect to a sequence defined in (a) or (b), or
(d) a sequence having at least 60% identity to a sequence as defined (a), (b) or (c).

17. Use of an activator, inhibitor or polynucleotide as defined in claim 14 in the manufacture of a medicament for the treatment of dyslipidaemia, coronary heart disease, atheroselerosis, thrombosis or obesity, angina, chronic renal failure, peripheral vascular disease, stroke, type II diabetes or metabolic syndrome (syndrome X).
